(19) United States
(12) Patent Application Publication
(10) Pub. No.: US 2009/0176042 A1
(43) Pub. Date: Jul. 9, 2009

(54) Medical Devices and EFAB Methods and Apparatus for Producing Them

(75) Inventor: Adam L. Cohen, Los Angeles, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica Inc.

(21) Appl. No.: 12/404,843

(22) Filed: Mar. 16, 2009

Related U.S. Application Data
(62) Division of application No. 10/697,598, filed on Oct. 29, 2003.
(60) Provisional application No. 60/422,007, filed on Oct. 29, 2002.

(51) Int. Cl. A61F 2/06; B05D 3/00

(52) U.S. Cl. 428/34.1; 427/2.1

(57) ABSTRACT

Various embodiments of the invention present miniature medical devices that may be formed totally or in part using electrochemical fabrication techniques. Sample medical devices include micro-tweezers or forceps, internally expandable stents, bifurcated or side branch stents, drug eluting stents, micro-valves and pumps, rotary ablation devices, electrical ablation devices (e.g. RF devices), micro-staplers, ultrasound catheters, and fluid filters. In some embodiments devices may be made out of a metal material while in other embodiments they may be made from a material (e.g. a polymer) that is molded from an electrochemically fabricated mold. Structural materials may include gold, platinum, silver, stainless steel, titanium or pyrolytic carbon-coated materials such as nickel, copper, and the like.

### RELATED APPLICATIONS

This application is a divisional of U.S. patent application Ser. No. 10/697,598 filed Oct. 29, 2003 which in turn claims benefit of U.S. Provisional Patent Application No. 60/422,007, filed Oct. 29, 2002, both of which are incorporated herein by reference as if set forth in full.

### FIELD OF THE INVENTION

The present invention relates generally to the field of miniature medical devices which may be formed by Electrochemical Fabrication.

### BACKGROUND

A technique for forming three-dimensional structures (e.g. parts, components, devices, and the like) from a plurality of adhered layers was invented by Adam L. Cohen and is known as Electrochemical Fabrication. It is being commercially pursued by Microfabrica® Inc. (formerly MEMGen® Corporation) of Burbank, Calif. under the name EFAB®. Certain variations of this technique were described in U.S. Pat. No. 6,027,630, issued on Feb. 22, 2000. The disclosed electrochemical deposition techniques allow the selective deposition of a material using a unique masking technique that involves the use of a mask that includes patterned conformable material on a support structure that is independent of the substrate onto which plating will occur. When desiring to perform an electrodeposition using the mask, the conformable portion of the mask is brought into contact with a substrate while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. (formerly MEMGen® Corporation) of Burbank, Calif. such masks have come to be known as INSTANT MASKS™ and the process known as INSTANT MASKING™ or INSTANT MASK™ plating. Selective depositions using conformable contact mask plating may be used to form single layers of material or may be used to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.
(3) A. Cohen, “3-D Micromachining by Electrochemical Fabrication”, Micromachine Devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFABTM)”, Chapter 19 of The MEMS Handbook, edited by Mohamed Gad-EI-Hak, CRC Press, 2002.
(9) “Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

The electrochemical deposition process may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate.
2. Then, blanket depositing at least one additional material by electrodeposition so that the additional deposit covers both the regions that were previously selectively deposited onto, and the regions of the substrate that did not receive any previously applied selective depositions.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to the immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated. At least one CC mask is needed for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of the substrate (or onto a previously formed layer or onto a previously deposited portion of a layer) on which deposition is to occur. The pressing together of the CC mask and substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6 separated from mask 8. CC mask plating selectively deposits material 22 onto a substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C. The CC mask plating process is distinct from a “through-mask” plating process in that in a through-mask plating process the separation of the masking material from the substrate would occur destructively. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the fabrication of the substrate on which plating is to occur (e.g. separate from a three-dimensional (3D) structure that is being formed). CC masks may be formed in a variety of ways, for example, a photolithographic process may be used. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing mask exposures. A first layer of a primary metal is electroplated onto an exposed plating base to fill a void in a photoresist, the photoresist is then removed and a secondary metal is electroplated over the first layer and over the plating base. The exposed surface of the secondary metal is then machined down to a height which exposes the first metal to produce a flat uniform surface extending across the both the primary and secondary metals. Formation of a second layer may then begin by applying a photoresist layer over the first layer and then repeating the process used to produce the first layer. The process is then repeated until the entire structure is formed and the secondary metal is removed by etching. The photoresist is formed over the plating base or previous layer by casting and the voids in the photoresist are formed by exposure of the photoresist through a patterned mask via X-rays or UV radiation.

Electrochemical Fabrication provides the ability to form prototypes and commercial quantities of miniature objects (e.g. mesoscale and microscale objects), parts, structures, devices, and the like at reasonable costs and in reasonable times. In fact, Electrochemical Fabrication is an enabler for the formation of many structures that were hitherto impossible to produce. Electrochemical Fabrication opens a new design and product spectrum in many industrial fields. Even though electrochemical fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art within the field or fields of a specific application.

### SUMMARY OF THE INVENTION

Objects and advantages of various aspects of the invention will be apparent to those of skill in the art upon review of the teachings herein.

In a first aspect of the invention, a process for forming a medical device, includes (a) forming and adhering a layer of material to a previously formed layer or to a substrate; (b) repeating the forming and adhering operation of (a) a plurality of times to build up a three-dimensional structure from a plurality of adhered layers; wherein the formation of at least a plurality of layers, comprises: (1) obtaining a selective pattern of deposition of at least a first material having voids, comprising at least one of: (a) selectively depositing at least a first material onto a substrate or previously formed layer such that voids remain; or (b) depositing at least a first material onto a substrate or previously formed layer and selectively etching the deposit of the first material to form voids therein; and (2) depositing at least a second material into the voids (c) after formation of a plurality of layers, removing at least one of the at least one first material or at least one second material to release the structure, wherein the structure comprises a medical device.

In a second aspect of the invention, a stent is provided with expansion capability provided by structural elements that transition from an orientation having a radial component to an orientation having less of a radial component.

In a third aspect of the invention, a stent capable of being at partially bifurcated is provided so that a first portion may extend along a first vessel and a second portion may extend along a second vessel and where a common portion extends along a vessel that joins the first and second vessels.

In a fourth aspect of the invention, a stent having struts is provided wherein at least a portion of the struts have pockets located therein with passages that extend from the pockets to a region outside the stent.

In a fifth aspect of the invention, a monolithically formed retriever includes a housing and a shaft moveable relative to the housing and further includes fingers that can be opened or closed by the relative movement of the shaft and the housing.

Further aspects of the invention will be understood by those of skill in the art upon reviewing the teachings herein. Other aspects of the invention may involve combinations of the above noted aspects of the invention. Other aspects of the invention may involve an apparatus that can be used in implementing the above method aspect of the invention. These other aspects of the invention may provide various combinations of the aspects presented above as well as provide other configurations, structures, functional relationships, and processes that have not been specifically set forth above.

### BRIEF DESCRIPTION OF THE DRAWINGS

FIGS. 1A-1C schematically depict side views of various stages of a CC mask plating process, while FIGS. 1(d)-(g) schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIG. 5 illustrates an example of a medical device according to a first embodiment of the invention.

FIGS. 6A-6B illustrate an example of a medical device according to a second embodiment of the invention.

FIG. 7 illustrates an example of a medical device according to another embodiment of the invention.

FIG. 8 illustrates an example of a medical device according to a further embodiment of the invention.

### DETAILED DESCRIPTION OF PREFERRED EMBODIMENTS OF THE INVENTION

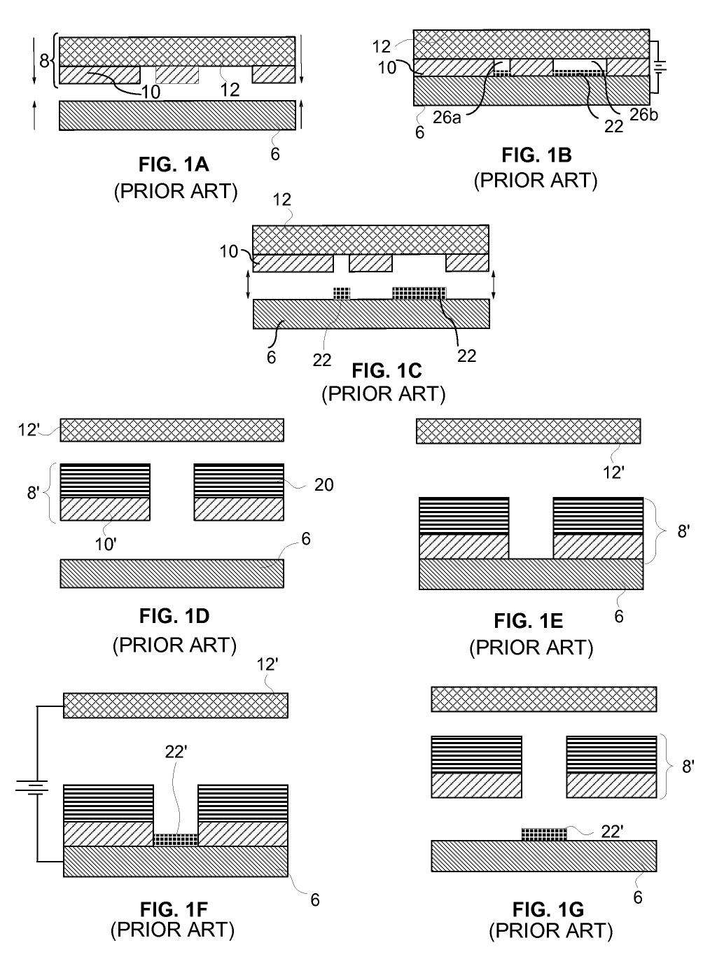

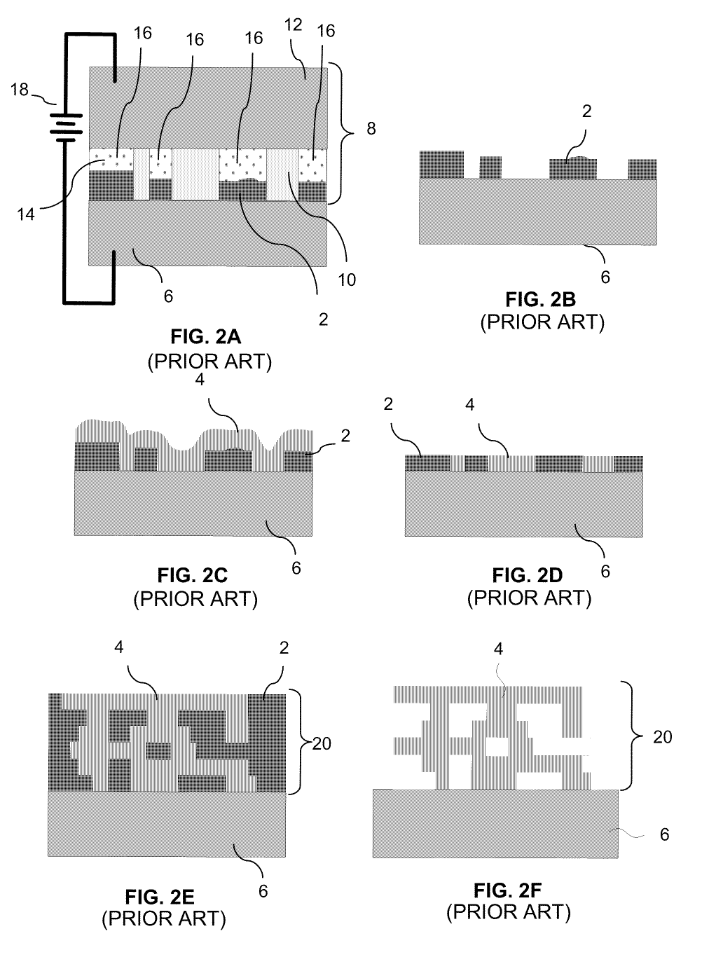

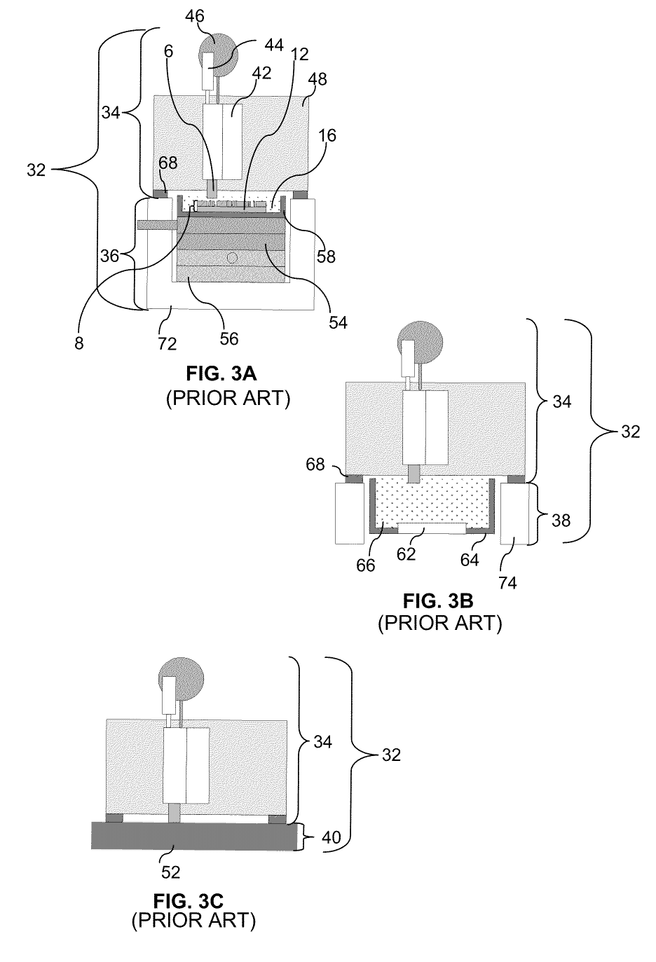

FIGS. 1A-1G, 2A-2F, and 3A-3C illustrate various features of one form of electrochemical fabrication that are known. Other electrochemical fabrication techniques are set forth in the '630 patent referenced above, in the various previously incorporated publications, in various other patents and patent applications incorporated herein by reference, still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

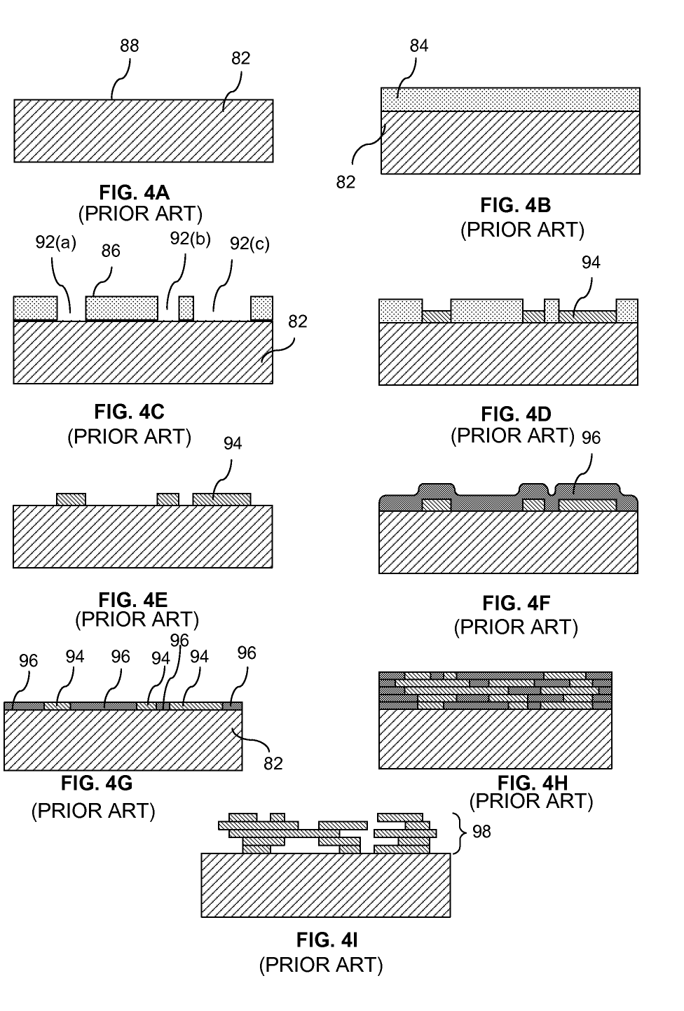

FIGS. 4A-4G illustrate various stages in the formation of a single layer of a multi-layer fabrication process where a second metal is deposited on a first metal as well as in openings in the first metal where its deposition forms part of the layer. In FIG. 4(a), a side view of a substrate 82 is shown, onto which patternable photoresist 84 is cast as shown in FIG. 4(b). In FIG. 4(c), a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4(d), a metal 94 (e.g. nickel) is shown as having been electroplated into the openings 92(a)-92(c). In FIG. 4(e), the photoresist has been removed (i.e. chemically stripped) from the substrate to expose regions of the substrate 82 which are not covered with the first metal 94. In FIG. 4(f), a second metal 96 (e.g., silver) is shown as having been blanket electroplated over the entire exposed portions of the substrate 82 (which is conductive) and over the first metal 94 (which is also conductive). FIG. 4(g) depicts the completed first layer of the structure which has resulted from the planarization of the first and second metals down to a height that exposes the first metal and sets a thickness for the first layer.

In FIG. 4(h) the result of repeating the process steps shown in FIGS. 4(b)-4(g) several times to form a multi-layer structure are shown where each layer consists of two materials. For most applications, one of these materials is removed as shown in FIG. 4(i) to yield a desired 3-D structure 98 (e.g. component or device).

The various embodiments, alternatives, and techniques disclosed herein may be used in combination with electrochemical fabrication techniques that use different types of patterning masks and masking techniques or even techniques that perform direct selective depositions without the need for masking. For example, conformable contact masks and masking operations may be used, proximity masks and masking operations (i.e. operations that use masks that at least partially selectively shield a substrate by their proximity to the substrate even if contact is not made) may be used, non-conformable masks and masking operations (i.e. masks and operations based on masks whose contact surfaces are not significantly conformable) may be used, and adhered masks and masking operations (masks and operations that use masks that are adhered to a substrate onto which selective deposition or etching is to occur as opposed to only being contacted to it) may be used.

Various embodiments present miniature medical devices that may be formed totally or in part using electrochemical fabrication techniques. These devices may be formed monolithically in an electrochemical fabrication process.

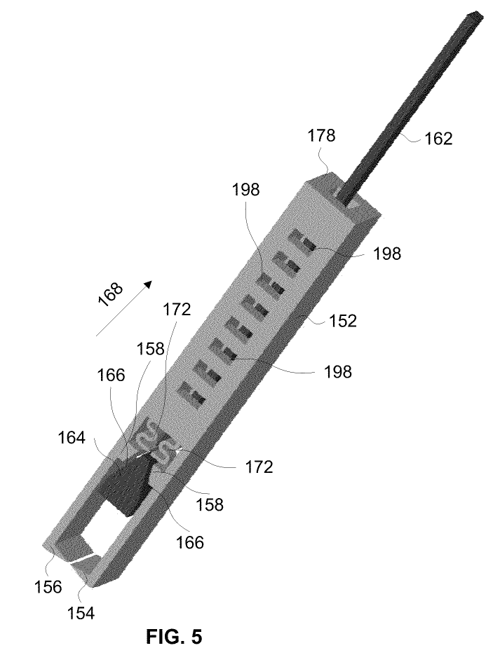

In a first embodiment a micro-tweezer or retriever is formed. The retriever is depicted in FIG. 5. The retriever includes a housing 152 with normally closed fingers 154 and 156. a control shaft 162 is located within housing 152 where it may have been formed. The shaft includes a tapered end element 164 that may be used to open normally closed fingers 154 and 156. When the tapered end element 164 is pulled in direction 168, relative to housing 152, contact between tapering surfaces 166 and 158 causes the fingers 154 and 156 to spread apart. Spring elements 172 resist surfaces 158 and 166 coming into contact and thereby cause surfaces 158 and 166 to separate when pressure along direction 168 is removed thus allowing normally closed fingers 154 and 156 to return to their gripping (i.e. closed) positions. Shaft 162 may be positioned within an extended housing that abuts the backend 178 of housing 152. During use, the extended housing may extend from the body of the patient and allow housing 152 to be held in position as shaft 162 is retracted. Alternatively, or additionally an extended covering may be located over housing 152 such that it covers the housing down to a position near the springs 172 and even partially covering the arms that hold fingers 154 and 156.

In some embodiments the retriever may take on a cylindrical shape as opposed to the rectangular shape illustrated. In the present embodiment holes 198 are shown in the sides of housing 152. These holes may be formed in the housing to aid in etching a sacrificial material from between the inner walls of the housing 152 and the shaft 162.

In the present embodiment the width of the housing may be under three French (i.e. under 1 millimeter) and even as small as 700 microns or less. Though the embodiment is shown with the fingers in a normally closed position, alternative embodiments may have fingers that are in a normally open position and that are closed by the movement of a shaft. In still other embodiments, fingers 154 and 156 may take on different shapes (e.g. they may include ridges or indentations), different number of fingers may be provided, and movement may be limited to a fraction of the fingers.

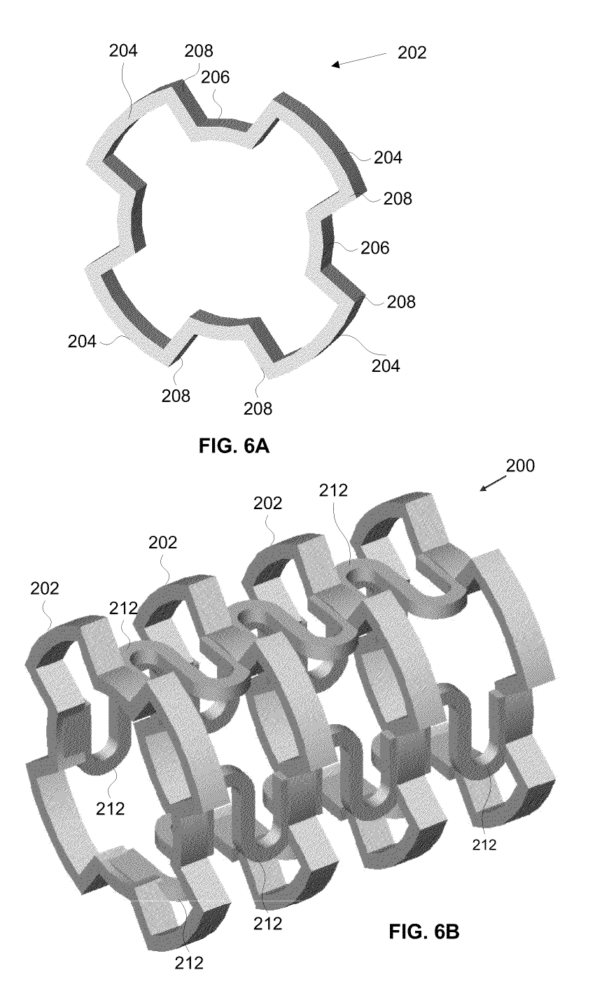

In a second embodiment of the invention an internally expandable stent is provided. An example of a stent according to the second embodiment is illustrated in the perspective views of FIGS. 6A and 6B. In a preferred embodiment the stent 200 (see FIG. 6B) includes a number of ring-like elements 202 where the ring-like elements include outer ring elements 204 and inner ring elements 206 connected by arms 208. The ring-like elements 202 are connected to adjacent ring-like elements with flexible S-shaped members 212. In the illustrated embodiment each ring-like member 202 is connected to an adjacent ring element 202 by four S-shaped members.

In alternative embodiments fewer S-shaped elements may be used. For example, two elements on opposite sides of each ring could be used or even one S-shaped element could be used to connect successive rings. In such embodiments, the S-shaped elements may all be located on the same side of successive rings or on opposite sides of successive rings or even in a spiraling pattern from along a chain of successive rings. In some embodiments the connecting members may extend from inner ring elements to outer ring elements. In some embodiments, S-shaped elements may be replaced by diamond shaped elements or the like. In other embodiments the ring-like elements 202 and the S-shaped elements 212 may be replaced by other structures that serve similar purposes (e.g. expansion and connection to form an extended structure).

After insertion into a vein, artery or other structure that is to be supported, a balloon or other device can be used to expand the diameter of the stent to its desired size. This may be accomplished by forcing inner ring elements 206 outward. As elements 206 move outward, arms 208 maybe be rotated from their radial orientations to circumferential orientations.

The stent of FIGS. 6(a) and 6(b) may be formed by electrochemical fabrication in its indicated configuration or alternatively the rings may be formed in more of a sheet-like manner and thereafter rolled into a cylindrical shape where joining segments of each ring may be connected together by bonding or alternatively by appropriate configuration of contacting elements such that interlocking can occur.

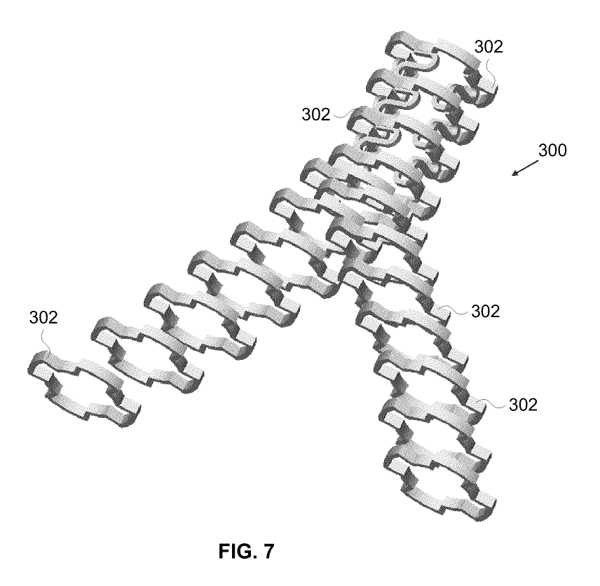

An additional embodiment of a stent is shown in FIG. 7 where a branching or bifurcated stent 300 is formed. In this embodiment two stents with connecting elements (not shown, e.g. S-shaped connecting elements) appropriately located on opposite sides of ring-like elements 302 (e.g. on one stent they may be located on the top and left side and on the other stent they may be located on the bottom and right side). The two stents could then be interlaced in a separable manner whereupon after insertion and meeting a fork in a vessel a portion of the ring-like elements could be separated from one another to allow the stent to be located along each branch of fork. After insertion into the vessel, a balloon could be used to expand one segment of the stent then the balloon could be moved to allow expansion of the other segment of the stent and finally the balloon could be moved to allow expansion of the overlapping portion of the stent.

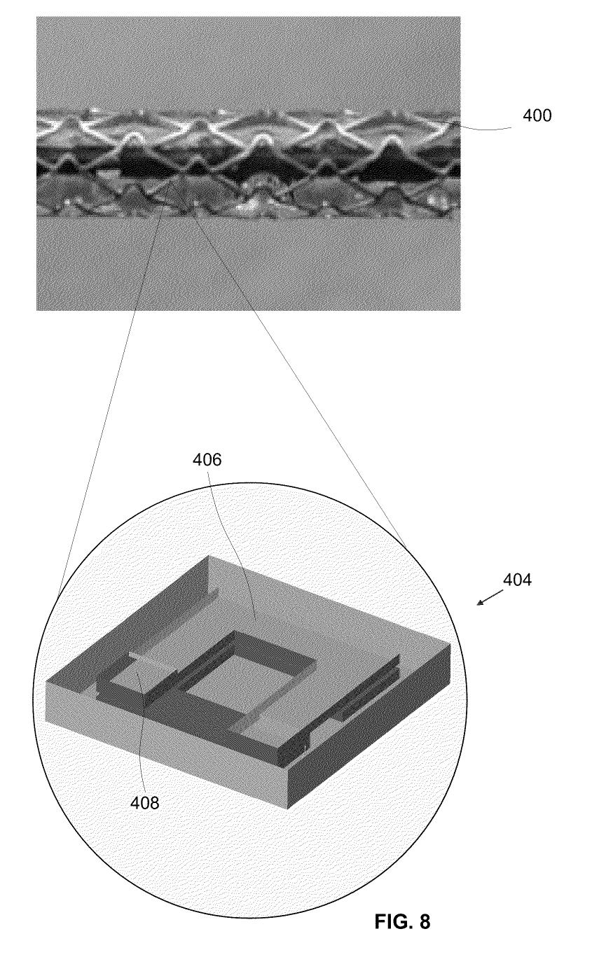

In an additional embodiment a stent may be formed where the struts of the stent (i.e. the connecting elements of the stent) may be formed from hollow structures as indicated in FIG. 8. FIG. 8 illustrates a small cross-sectional portion of one of the struts (i.e. arms or connecting elements of the stent). A stent 400 is shown as having a number of diamond shaped connecting arms or struts where according to this embodiment some of the struts may be formed such that they include perimeter elements 404. Passages 408 may extend from an interior region 406 to regions external to the stent.

Such hollow regions 406 may be filled with a desired drug that could be slowly dispensed to the patient via the passages 408. The hollow interior regions 406 of the struts may take the form of multi-layer spiral pores or alternatively they may run the length of the struts with small apertures allowing access to the drug.

In still other embodiments electrochemical fabrication may be used to form reusable or sacrificial mold structures that may be used in forming medical devices from polymer materials. Such use of electrochemical fabrication is set forth in one of the applications incorporated herein by reference.

In still other embodiments, microvalves and/or pumps may be created. Ablation tools may also be formed. These tools may include spinning blades that may be rotated on a shaft or by a motor that is built by electrochemical fabrication during the formation of the rest of the ablation device. Other embodiments may provide microstaplers and in still other embodiments ultrasound catheters may be formed with transducers that are formed via electrochemical fabrication along with motors that may be used to rotate or oscillate them. In still other embodiments filters may be formed that may be inserted into blood vessels.

Various other embodiments of the present invention exist. Some of these embodiments may be based on a combination of the teachings explicitly set forth herein with various teachings incorporated herein by reference. Some embodiments may not use any blanket deposition process and/or they may not use a planarization process. Some embodiments may involve the selective deposition of a plurality of different materials on a single layer or on different layers. Some embodiments may use blanket depositions processes that are not electrodeposition processes. Some embodiments may use selective deposition processes on some layers that are not conformable contact masking processes and are not even electrodeposition processes. Some embodiments may use nickel as a structural material while other embodiments may use different materials such as gold, silver, or any other electrodepositable materials that can be separated from copper and/or some other sacrificial material. Some embodiments may use copper as the structural material with or without a sacrificial material. Some embodiments may remove a sacrificial material while other embodiments may not. In some embodiments the anode may be different from the conformable contact mask support and the support may be a porous structure or other perforated structure. Some embodiments may use multiple conformable contact masks with different patterns so as to deposit different selective patterns of material on different layers and/or on different portions of a single layer.

In view of the teachings herein, many further embodiments, alternatives in design and uses of the instant invention will be apparent to those of skill in the art. As such, it is not intended that the invention be limited to the particular illustrative embodiments, alternatives, and uses described above but instead that it be solely limited by the claims presented hereafter.

I claim:

1. A monolithically formed retriever comprising a housing and a shaft moveable relative to the housing and comprising fingers that can be opened or closed by the relative movement of the shaft and the housing.

2. The device of claim 1 formed by a process, comprising:
(a) forming and adhering a layer of material to a previously formed layer or to a substrate;
(b) repeating the forming and adhering operation of (a) a plurality of times to build up a three-dimensional structure from a plurality of adhered layers;
wherein the formation of at least a plurality of layers, comprises:
(1) obtaining a selective pattern of deposition of at least a first material having voids, comprising at least one of:
(a) selectively depositing at least a first material onto a substrate or previously formed layer such that voids remain; or
(b) depositing at least a first material onto a substrate or previously formed layer and selectively etching the deposit of the first material to form voids therein; and
(2) depositing at least a second material into the voids
(c) after formation of a plurality of layers, removing at least one of the at least one first material or at least one second material to release the structure,
wherein the structure comprises a medical device.
